(19) United States
(12) Patent Application Publication
(10) Pub. No.: US 2025/0302448 A1
(43) Pub. Date: Oct. 2, 2025

(54) SETTINGS PROGRESSION FOR ULTRASOUND

(71) Applicant: KONINKLIJKE PHILIPS N.V., EINDHOVEN (NL)

(72) Inventors: Shyam Bharat, Arlington, MA (US); Balasundar Raju, North Andover, MA (US); Raghavendra Srinivasa Naidu, Auburndale, MA (US)

(21) Appl. No.: 18/862,279

(22) PCT Filed: Apr. 21, 2023
(86) PCT No.: PCT/EP2023/060451

§ 371 Date: Nov. 1, 2024

Related U.S. Application Data
(60) Provisional application No. 63/337,362, filed on May 2, 2022.

(51) Int. Cl. A61B 8/00

(52) U.S. Cl. CPC A61B 8/54; A61B 8/463; A61B 8/585

(57) ABSTRACT

An ultrasound system includes an ultrasound probe, a display, and an ultrasound base. The ultrasound probe transmits ultrasound imaging beams in accordance with combinations of settings. The display displays images based on feedback generated from the ultrasound imaging beams. The ultrasound base is interfaced with the ultrasound probe and the display. The ultrasound system is configured to automatically progress through a plurality of combinations of settings for transmitting the ultrasound imaging beams.

### BACKGROUND

Ultrasound use for lung evaluation has increased in recent years due to the COVID pandemic. Several features/artifacts which may be detected on ultrasound are indicative of lung disease severity. Examples of such features/artifacts include B-lines, pleural line patterns, consolidation, lung sliding etc. Recent studies have shown that the manifestation of these features/artifacts on ultrasound can vary with the specific ultrasound system settings chosen for transmission of ultrasound beams. For example, both the quality and quantity of B-lines have been found to vary with system settings such as focal depth, a harmonics setting, image gain, TGC etc. Therefore, variations in system settings have the potential to indirectly lead to differing diagnostic conclusions. For any of a variety of reasons, results of ultrasound imaging sometimes are sometime not optimal due to usage of combinations of system settings which are not optimal.

### SUMMARY

According to an aspect of the present disclosure, an ultrasound system includes an ultrasound probe, a display and an ultrasound base. The ultrasound probe transmits ultrasound imaging beams in accordance with combinations of settings. The display displays images based on feedback generated from the ultrasound imaging beams. The ultrasound base is interfaced with the ultrasound probe and the display. The ultrasound system is configured to automatically progress through a plurality of combinations of settings for transmitting the ultrasound imaging beams.

According to another aspect of the present disclosure, a method of operation for an ultrasound system includes transmitting, by an ultrasound probe, a first ultrasound imaging beam in accordance with a first combination of settings; displaying, by a display, a first image based on feedback generated from the first ultrasound imaging beam; transmitting, by the ultrasound probe, a second ultrasound imaging beam in accordance with a second combination of settings; displaying, by the display, a second image based on feedback generated from the second ultrasound imaging beam; and automatically progressing through a plurality of combinations of settings including the first combination of settings and the second combination of settings.

According to another aspect of the present disclosure, a controller includes a memory that stores instructions; and a processor that executes the instructions. When executed by the processor, the instructions cause the controller to: control transmission, by an ultrasound probe, of a first ultrasound imaging beam in accordance with a first combination of settings in an automated progression; control display, by a display, of a first image based on feedback generated from the first ultrasound imaging beam; control transmission, by the ultrasound probe, of a second ultrasound imaging beam in accordance with a second combination of settings in the automated progression; control display, by the display, of a second image based on feedback generated from the second ultrasound imaging beam; and automatically progress through a plurality of combinations of settings for transmitting a plurality of ultrasound imaging beams including the first ultrasound imaging beam and the second ultrasound imaging beam.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a system for settings progression for ultrasound, in accordance with a representative embodiment.

FIG. 1B illustrates another system for settings progression for ultrasound, in accordance with a representative embodiment.

FIG. 2 illustrates a method for settings progression for ultrasound, in accordance with a representative embodiment.

FIG. 3 illustrates a user interface for settings progression for ultrasound, in accordance with a representative embodiment.

FIG. 4 illustrates another method for settings progression for ultrasound, in accordance with a representative embodiment.

FIG. 5 illustrates another user interface for settings progression for ultrasound, in accordance with a representative embodiment.

FIG. 6 illustrates another user interface for settings progression for ultrasound, in accordance with a representative embodiment.

FIG. 7 illustrates a computer system, on which a method for settings progression for ultrasound is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims.

As described herein, an automated approach provides standardized and consistent interpretation of lung ultrasound features. The systems and methods described herein may automatically progress through combinations of ultrasound system settings to evaluate the impact of changing settings on the manifestation of one or more ultrasound features such as B-lines. As an initial example use, the results may be output via a user interface to enable users to pick desired combination of settings that yield a particular manifestation of the one or more ultrasound features.

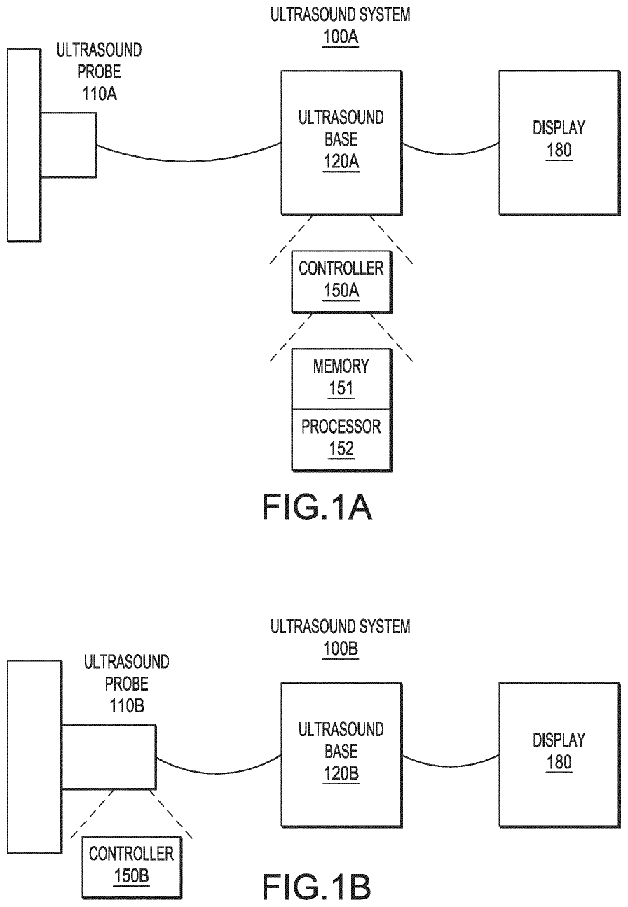

FIG. 1A illustrates a system 100 for settings progression for ultrasound, in accordance with a representative embodiment.

The ultrasound system 100A in FIG. 1A is a system for settings progression for ultrasound and includes components that may be provided together or that may be distributed. The ultrasound system 100A includes an ultrasound probe 110A, an ultrasound base 120A, and a display 180. The ultrasound base 120A includes a controller 150A, and the controller 150A includes a memory 151 and a processor 152.

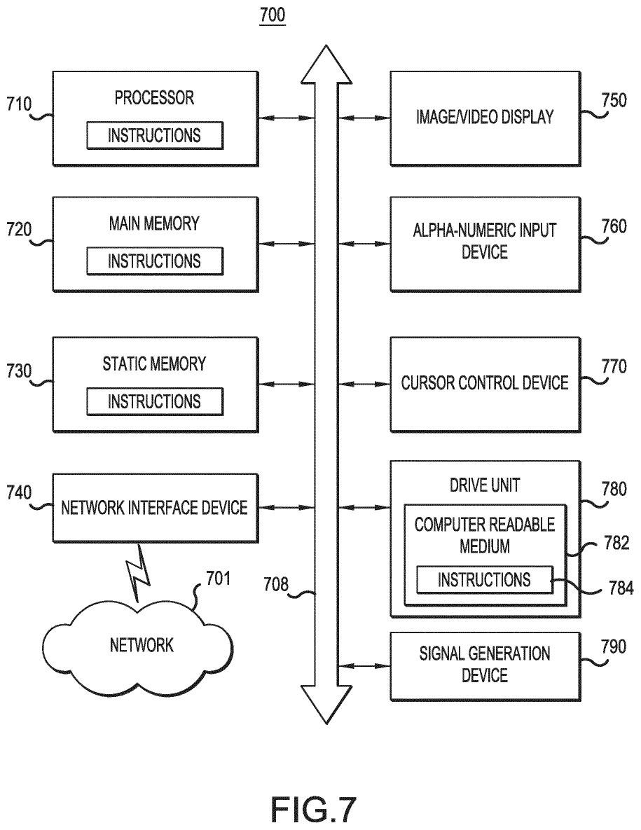

The ultrasound base 120A may comprise an ultrasound cart, a mobile computer such as a tablet computer or laptop used for controlling ultrasound procedures and processing results, or even a stationary computer system used for controlling ultrasound procedures and processing results. The ultrasound base 120A is configured for use to control ultrasound procedures and process feedback from ultrasound imaging beams transmitted from the ultrasound probe 110A. A computer that can be used to implement the ultrasound base 120A is depicted in FIG. 7, though an ultrasound base 120A may include more or fewer elements than depicted in FIG. 1A or FIG. 7.

The controller 150A in the ultrasound base 120A in FIG. 1A may perform functionality attributed to a controller as described herein. The memory 151 stores instructions and the processor 152 executes the instructions. In some embodiments, multiple different elements of the ultrasound system 100A in FIG. 1A may include a controller such as the controller 150A. For example, the ultrasound probe 110A, the ultrasound base 120A and the display 180 may each include separate controllers with memories that store instructions and processors that execute the instructions. The controller 150A may perform some of the operations described herein directly and may implement other operations described herein indirectly. For example, the controller 150A may indirectly control other operations such as by generating and transmitting content to be displayed on the display 180. The controller 150A may directly control other operations such as logical operations performed by the processor 152 executing instructions from the memory 151 based on input received from electronic elements and/or users via the interfaces. Accordingly, the processes implemented by the controller 150A when the processor 152 executes instructions from the memory 151 may include steps not directly performed by the controller 150A. One example of operations performed by the controller 150A is that the controller 150A is configured to automatically progress through a plurality of combinations of settings for transmitting the ultrasound imaging beams, even though the ultrasound imaging beams are transmitted by the ultrasound probe 110A.

The controller 150A may also include interfaces, such as a first interface, a second interface, a third interface, and a fourth interface. One or more of the interfaces may include ports, disk drives, wireless antennas, or other types of receiver circuitry that connect the controller 150A to other electronic elements. One or more of the interfaces may also include user interfaces such as buttons, keys, a mouse, a microphone, a speaker, a display separate from the display 180, or other elements that users can use to interact with the controller 150A such as to enter instructions and receive output.

The display 180 may be local to the ultrasound base 120A or may be remotely connected to the ultrasound base 120A. The display 180 may be connected to the ultrasound base 120A via a local wired interface such as an Ethernet cable or via a local wireless interface such as a Wi-Fi connection. The display 180 may be interfaced with other user input devices by which users can input instructions, including mouses, keyboards, thumbwheels and so on. The display 180 may be a monitor such as a computer monitor, a display on a mobile device, an augmented reality display, a television, an electronic whiteboard, or another screen configured to display electronic imagery. The display 180 may include one or more input interface(s) such as those noted above that may connect to other elements or components, as well as an interactive touch screen configured to display prompts to users and collect touch input from users.

User interfaces for the ultrasound system 100A may include a touch user interface on the display 180, mouses, keyboards and thumbwheels, control elements on the ultrasound probe 110A, and more. The user interfaces may be used to collect user input regarding the range of values for each parameter in the combinations of settings. The user interfaces may also be used to guide the user through the image acquisition process. For example, the display 180 may display instructions for when to hold the ultrasound probe 110A steady. The user interfaces may include a progress bar on the display 180 or on the ultrasound probe 110A showing completion of a progression as a percentage, for example. The results of the progression of settings may be presented to the user on the display 180 as a set of ultrasound images, so that the user may pick a desired combination of settings that yields a particular manifestation of the ultrasound features corresponding to one or more of the set of ultrasound images.

FIG. 1B illustrates another system for settings progression for ultrasound, in accordance with a representative embodiment.

In the ultrasound system 100B of FIG. 1B, the controller 150B is implemented as an element of the ultrasound probe 1101B instead of the ultrasound base 120B. The controller 150B in FIG. 1B may perform functionality attributed to a controller as described herein. The controller 150B may include a memory that stores instructions and a processor that processes the instructions as described already with respect to FIG. 1A. One example of operations performed by the controller 150B is that the controller 150B is configured to automatically progress through a plurality of combinations of settings for transmitting the ultrasound imaging beams, and the ultrasound probe 110B that includes the controller 150B transmits the ultrasound imaging beams. Other than implementing the controller 150B in the ultrasound probe 110B rather than the ultrasound base 120B, the features of the ultrasound system 100B may be similar or identical to the features of the ultrasound system 100B in FIG. 1A, and repeated descriptions thereof are omitted for brevity.

Using the ultrasound system 100A or the ultrasound system 100B, settings progression for ultrasound may automatically progress through sequences of combinations of ultrasound system settings to evaluate the impact of changing settings on the manifestation of features, including but not limited to B-lines. For each settings combination, the ultrasound system 100A or the ultrasound system 100B may store image(s) to capture the manifestation of the feature(s). Upon obtaining confirmation, the ultrasound system 100A or the ultrasound system 100B may store the images corresponding to the desired setting combination. In some embodiments, the user may be enabled to transfer selected ultrasound images to an image storage database provided as part of the ultrasound base 120A, the ultrasound base 120B or as a separate element.

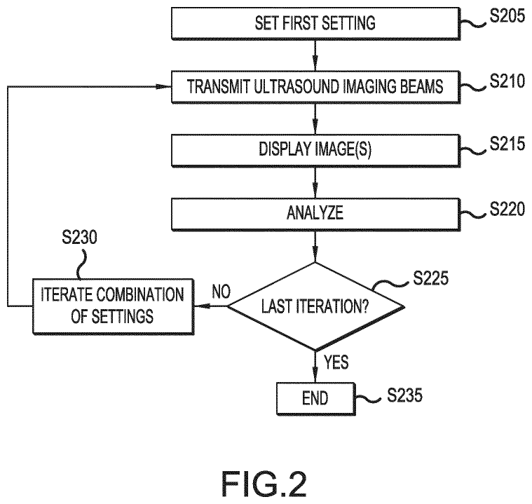

FIG. 2 illustrates a method for settings progression for ultrasound, in accordance with a representative embodiment.

The method of FIG. 2 may be performed by the ultrasound system 100A in FIG. 1A or by the ultrasound system 100B in FIG. 1B.

At S205, a first setting is set. For example, a first combination of settings may be set at S205. The first combination of settings may be the first combination in a progression of combinations, such as a first combination of settings, a second combination of settings, a third combination of settings, a fourth combination of settings, and so on. Additionally, combinations of settings may be cyclical and iterative so that progressions through the combinations of settings may be repeated in the same sequence and with the same combinations each time the ultrasound probe 110A or the ultrasound probe 110B is activated to transmit the ultrasound imaging beam(s) during an ultrasound session. Also, or alternatively, combinations of settings may be dynamically adaptable so that progressions through the combinations of settings may be varied to have different sequences in different combinations one or more times the ultrasound probe 110A or the ultrasound probe 110B is activated to transmit the ultrasound imaging beam(s) during an ultrasound session. In some embodiments, the range of parameter values for a setting or more than one setting may be adaptively updated, such as when no images produced in a progression satisfy minimum quality thresholds.

At S210, one or more ultrasound imaging beam(s) are transmitted. The ultrasound imaging beam(s) are transmitted at S210 based on the combination of settings set at S205. Each combination of settings may include a first setting for image depth, a second setting for harmonics a third setting for a gain as a function of image depth, and a fourth setting for focal depth. Other setting may also, or alternatively, be used in the combination of settings set at S205. For example, a fifth setting for overall gain may be set and/or a sixth setting for transmit frequency may be set. Transmit frequency may refer to a center frequency at the center of a transmission band used for transmitting the ultrasound imaging beam(s).

At S215, one or more image(s) are displayed based on the one or more ultrasound imaging beam(s) transmitted at S210. The one or more image(s) may be displayed on a display 180. In some embodiments, the display 180 may be configured to simultaneously display a first image based on feedback from a first combination of settings and a second image based on feedback from a second combination of settings. More than two images from two or more than two combinations of settings may be simultaneously displayed for ease of comparison by a user.

At S220, an analysis is performed based on the data used to generate and display the one or more image(s) displayed at S215. The analysis may include counting the number of B-lines and analyzing the quality of B-lines present in the data used to generate and display the one or more image(s) displayed at S215. The number and quality of B-lines may reflect the appropriateness of the combination of settings used to generate and transmit the one or more ultrasound imaging beam(s) at S210.

B-lines are lung ultrasound (LUS) artifacts consisting of vertical lines originating at the pleura and extending to the far field of the image. B-lines are characteristic of common lung pathologies including cardiogenic pulmonary edema, interstitial lung disease, acute respiratory distress syndrome, pulmonary fibrosis, and others. B-line assessment has become a core part of point-of-care ultrasound (POCUS) in emergency medicine, critical care, family medicine, internal medicine, and many subspecialties.

B-line images depend on technical aspects of image acquisition, and are one of the reasons combinations of settings may be used to determine optimal settings. Quantification of B-lines provides a tool to evaluate the severity of pulmonary edema and a dynamic measure of progression of disease. However, B-line quantification is subject to variability based on a number of factors such as sonographer expertise level, time spent in evaluation, the type of transducer, image depth setting used, focal depth and other patient-related factors. Variation may be partially mitigated by using automated quantification solutions. However, such automation is still reliant on the underlying image quality, which in turn may be impacted by the chosen ultrasound system settings.

At S225, a determination is made as to whether the ultrasound imaging beam(s) transmitted at S210 were transmitted based on the last combination of settings. The determination at S225 may simply identify whether the most recent combination of settings is the last combination in a sequence.

If the ultrasound imaging beam(s) transmitted at S210 were not based on the last combination of settings, (S225=No), at S230 the method of FIG. 2 includes incrementing the combination of settings to the next combination of settings and then returning to S210. For example, combinations of settings may be stored sequentially in a memory so that the method progresses through a sequence of combinations. In another example, a library of combinations may include entries associated with unique identifications, and the method may refer to a list of a subset of unique identifications to identify each combination to use. In the latter example, sequences of combinations to use may be adaptable, so that even during a single session the sequence may be updated to add or remove unique identifications for combinations to use or not use.

If the ultrasound imaging beam(s) transmitted at S210 were based on the last combination of settings (S225=Yes), at S235 the method of FIG. 2 ends. Although not shown, the method of FIG. 2 may include a determination of which combination of settings resulted in optimal data based on transmitted ultrasound beams. The determination may be made by a clinician, or by the ultrasound system 100A or the ultrasound system 100B. For example, the ultrasound system 100A may “blindly” proceed through a progression of different settings, and then apply an automated parameter detection algorithm to determine which setting works best. An automated parameter detection algorithm may leverage an image quantification program, such as by determining which ultrasound images include 4 or more B-lines. An automated parameter detection algorithm may also parameterize a quality of features such as B-lines, such as by ensuring that B-lines include characteristics such as average brightness above a predetermined threshold.

Additionally, the combinations of settings may vary for different pathologies. Users may be provided a choice to select the best setting for diagnostic measurement/presentation for each different pathology. In some embodiments, a user may specify the pathology, and the ultrasound system 100A or the ultrasound system 100B may select the combinations of settings to use in a progression based on the specified pathology.

In the method of FIG. 2, automated quantification solutions for ultrasound systems such as the ultrasound system 100A and the ultrasound system 100B may quantify, for example, B-lines to provide a seamless workflow for users. Once the ultrasound probe 110A or the ultrasound probe 110B is positioned at a lung zone/location, the ultrasound system 100A or the ultrasound system 100B may choose and set a certain first combination of system settings at S205, initiate the automated quantification solution(s), transmit the ultrasound imaging beams at S210, display the ultrasound image(s) at S215, analyze the data used to generate the ultrasound image(s) at S220, log the results, and then progress to the next combination of settings and repeat the process. Once all combinations have been achieved (S225=Yes), a user may be prompted to move to the next scanning location. The combination of the settings progression for ultrasound and B-lines quantification features may increase robustness and improve flexibility of lung solutions to accommodate user preferences. At any point, the user may be enabled to review the results as a function of the setting combination (e.g., a first combination, a second combination etc.). Alternately, the user may be enabled to complete scanning of, for example, the lung(s), and review the results at the end. The final choice of settings may rest with the user. The quantification results corresponding to the chosen setting combination may be automatically sent to the imaging report.

In some embodiments, the settings progression for ultrasound may be implemented as a software update that runs on an ultrasound system 100A or an ultrasound system 100B.

An example of a progression of combinations of settings is set forth below in Table 1:

TABLE 1

| | | | | | |
|---|---|---|---|---|---|
| Combination1 | Setting1A | Setting2A | Setting3A | Setting4A | Setting5A |
| Combination2 | Setting1B | Setting2B | Setting3B | Setting4B | Setting5B |
| Combination3 | Setting1C | Setting2C | Setting3C | Setting4C | Setting5C |
| Combination4 | Setting1D | Setting2D | Setting3D | Setting4D | Setting5D |
| Combination5 | Setting1E | Setting2E | Setting3E | Setting4E | Setting5E |
| Combination6 | Setting1F | Setting2F | Setting3F | Setting4F | Setting5F |
| Combination7 | Setting1G | Setting2G | Setting3G | Setting4G | Setting5G |
| Combination8 | Setting1H | Setting2H | Setting3H | Setting4H | Setting5H. |

As shown in Table 1 above, eight combinations for five different settings may be used for a progression of combinations of settings. As an example, the first setting may be for image depth, the second setting may be for harmonics, the third setting may be for a gain as a function of image depth, and the fourth setting may be for focal depth, the fifth setting may be for another parameter which may be variably set. The fifth setting may be, for example, a setting for overall gain or a setting for transmit frequency. Other setting may also, or alternatively, be used in the combination of settings in Table 1.

In Table 1, one or more of the settings may retain the same value in each combination while one or more other settings may vary between combinations. Additionally, while Table 1 may be established in advance of a progression, in some embodiments some settings may be adaptively changed after the progression starts, such as if the controller 150A or the controller 150B recognizes that one or more settings are consistently not being used in combinations that result in ultrasound images with satisfactory characteristics (e.g., number and quality of B-lines). Moreover, while eight combinations of settings are shown in Table 1, a progression may include more or fewer than eight combinations of settings.

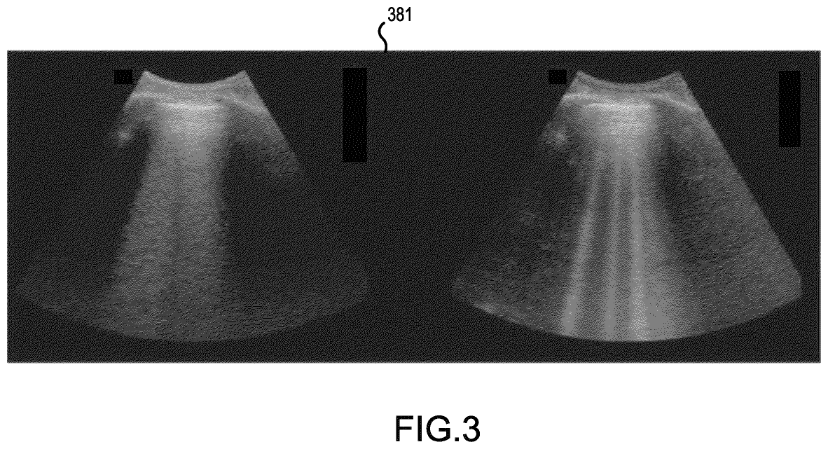

FIG. 3 illustrates a user interface for settings progression for ultrasound, in accordance with a representative embodiment.

FIG. 3 shows an example comparison between B-lines resulting from two combinations of ultrasound settings, on a patient presenting with shortness of breath. Notably, in FIG. 3 different manifestations of B-lines (both qualitatively and quantitatively) may be observed between the 2 image setting combinations. Examples of the settings that lead to the variations in the two images in FIG. 3 include focal depth, a harmonics setting, image gain, time gain compensation (TGC), overall gain and/or transmit frequency. As should be evident, changes in combinations of system settings have the potential to indirectly lead to differing diagnostic conclusions, and this is addressed by the settings progression for ultrasound described herein.

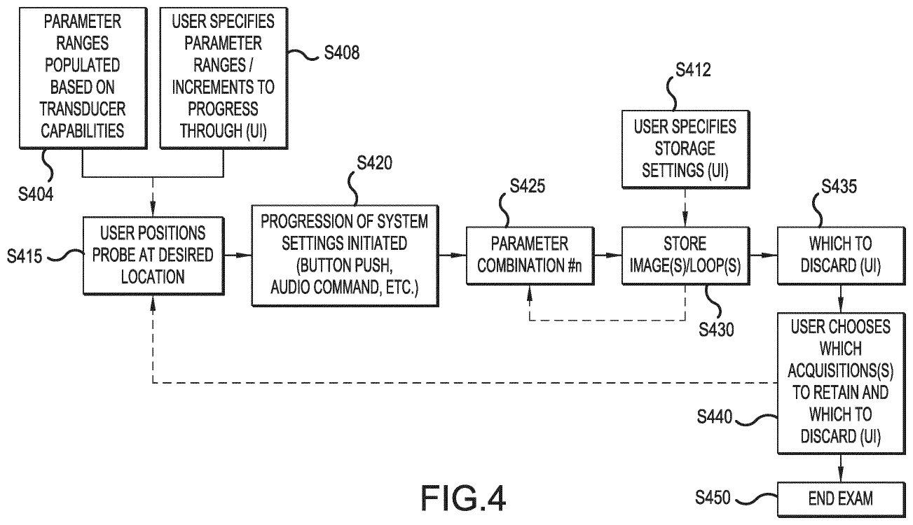

FIG. 4 illustrates another method for settings progression for ultrasound, in accordance with a representative embodiment.

The method of FIG. 4 starts at S404 with populating ranges of parameter values based on transducer capabilities. The parameter ranges may be ranges of parameter values to which each of a plurality of settings may be set. Ranges may be as simple as binary ranges with values limited to 0 and 1, Yes and No, or On and Off. Ranges may include larger numbers of potential values, such as 0 to 100 or A to Z.

At S408, the method of FIG. 4 includes specifying ranges of parameter values and increments to progress through. The parameter ranges and increments may be specified through a user interface as shown in FIG. 4. However, parameter ranges and increments may also be preset, such as by an entity that provides settings progression for ultrasound as a software update for users. Some parameter ranges and increments may not be amendable to change, such as parameter ranges that are binary so that the only possible values are 0 and 1, Yes and No, or On and Off. Additionally, in embodiments where a user may specify the parameter ranges and increments, a default combination of values may be provided initially or after a period of use, so that the user is provided an option to change the parameter ranges and increments.

At S412, storage settings are specified. For example, a user may input storage settings via a user interface on the display 180, or via a user input device such as a mouse, keyboard, thumbwheel, button or another type of user interface. The storage setting may specify which images generated by different combinations of settings are to be stored, such as for simultaneous or subsequent display on the display 180. For example, storage settings may specify that only ultrasound image showing four or more B-lines should be stored and displayed, since not all combinations of settings will result in 4 or more B-lines in images.

At S415, the probe is positioned at a desired location. For example, a clinician may move the ultrasound probe 110A or the ultrasound probe 110B to a desired location.

At S420, the progression of combinations of settings for the ultrasound system 100A or the ultrasound system 100B is initiated. For example, the clinician may push a button to initiate transmission of ultrasound imaging beams at each of a predetermined sequence of combinations of settings.

A loop sequence for the progression of combinations of settings is performed at S425 and S430. Specifically, at S425, parameter combination #n is used to generate and transmit one or more ultrasound beam(s), and at S430, the resulting image(s) are stored. The images stored at S430 are stored in accordance with the storage settings specified at S412. In some embodiments, all ultrasound images are stored or at least may be stored. In other embodiments, storage may be limited to a subset of the ultrasound images, such as images showing four or more B-lines.

At S435, the method of FIG. 4 includes determining which images to discard. For example, images which are stored at S430 according to the storage settings at S412 may be discarded according to a predetermined determination or instruction at S435. As an example, a set of ultrasound images each based on a different combination of settings may be displayed on the display 180 together, or in sequence, and the ultrasound system 100A or the ultrasound system 100B may execute the instructions to and delete certain ultrasound images and retain other ultrasound images.

In some embodiments, the method of FIG. 4 may use determinations at S430 and S435 for setting a default combination of settings based on selections of images. The images are generated from automatically progressing through the plurality of combinations of settings, and the ultrasound system 100A and the ultrasound system 100B may recognize patterns of selections of images and corresponding combinations to identify defaults for users to start with for different pathologies.

At S440, the method of FIG. 4 includes accepting a choice of which acquisitions to retain and which to discard. For example, a user may be presented one or more ultrasound images from each of a set of combinations of settings, such as ultrasound images from 10 different combinations of settings. The user may be allowed to select which of the ultrasound images reflect the optimal results, such as by which of the ultrasound images show four or more B-lines with the best image quality.

The selection of images at S440 may be used to adjust the combinations of settings used in the progression by the ultrasound system 100A or the ultrasound system 100B. For example, if a user repeatedly discards images from a particular combination, the ultrasound system 100A or the ultrasound system 100B may stop including the particular combination in the progression, and may replace the particular combination with another combination of settings.

As set forth above with respect to the method of FIG. 4, a user may position the ultrasound probe 110A or the ultrasound probe 110B at a certain lung zone/location, and the ultrasound system 100A or the ultrasound system 100B may choose a certain combination of system settings to start a progression. The parameter ranges for the settings may be determined based on the capabilities of the selected transducer, such as such as frequency, bandwidth etc. In some embodiments, the choice of settings may also be guided by an initial choice of parameter ranges made by the user. As an example of settings in a progression, a first setting may include a frequency of 2 MHz, focused at 6 centimeters, and a second setting may include a frequency of 5 Megahertz, focused at 3 centimeters.

The ultrasound system 100A and the ultrasound system 100B may integrate settings progression for ultrasound with quantification for the settings, such as by automating the counting of B-lines and the assessment of quality based on a quantification program, so that determinations for which images to retain at S430, S435 and S440 are based fully or partly on the counting.

In some embodiments, a program for settings progression for ultrasound may communicate or otherwise interact with an automated quantification solution that exists separately on the ultrasound system 100A or the ultrasound system 10)B, to initiate auto-quantification and log the results.

In the method of FIG. 4, the progression(s) are repeated each time the user moves the ultrasound probe 110A or the ultrasound probe 1101B to a desired probe position. Once all combinations have been achieved, such as after several seconds such as 5 seconds, the user may be prompted to move to the next scanning location. In some embodiments, the user may be enabled to review the results as a function of each setting combination such as a first combination or a second combination, so repeatedly during a single progression. In some other embodiments, the user may complete scanning the lung(s) and review the results at the end of the progression. The final choice of settings may be set by the user, and the quantification results corresponding to the chosen setting combination may be automatically used to generate a final imaging report for an ultrasound session.

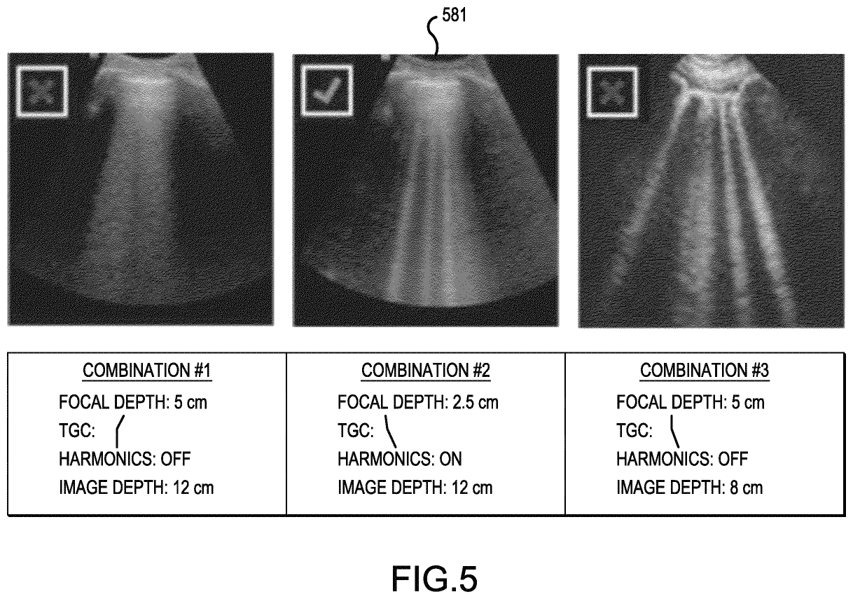

FIG. 5 illustrates another user interface for settings progression for ultrasound, in accordance with a representative embodiment.

In FIG. 5, a user interface 581 allows a user to choose optimal result. The B-mode images in FIG. 5 may be representative images. The consolidated results from a given probe position may be presented to the user via the user interface 581 shown in FIG. 5. The user may choose the result deemed to be optimal based on their clinical expertise, and the result may be logged by the system. The system may then either remember the choice of settings and use that to log results from some or all other probe positions, or alternately the user may pick a different choice of settings for other probe positions.

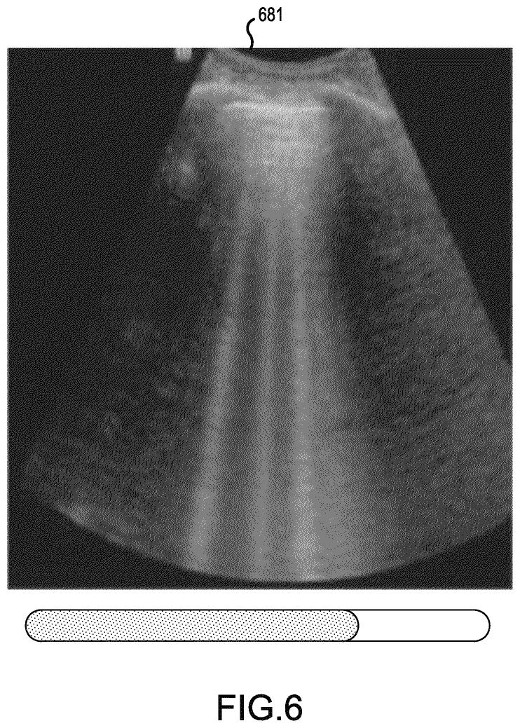

FIG. 6 illustrates another user interface for settings progression for ultrasound, in accordance with a representative embodiment.

The user interface 681 shown in FIG. 6 includes a progress bar for each probe position. The B-mode image on the user interface 681 may be a representative image. The user interface 681 includes the progress bar as an additional component that tracks the progress of the scan at each location. Presenting the progress bar to the user may enable the user to know how much longer they need to hold the probe at the location before all acquisitions are complete. Typically, all acquisitions at a given probe position may be completed in a few seconds, though the length of time required to complete all acquisitions may depend also on the length of the loop desired by the user for each combination of settings.

FIG. 7 illustrates a computer system, on which a method for settings progression for ultrasound is implemented, in accordance with another representative embodiment.

Referring to FIG. 7, the computer system 700 includes a set of software instructions that can be executed to cause the computer system 700 to perform any of the methods or computer-based functions disclosed herein. The computer system 700 may operate as a standalone device or may be connected, for example, using a network 701, to other computer systems or peripheral devices. In embodiments, a computer system 700 performs logical processing based on digital signals received via an analog-to-digital converter.

In a networked deployment, the computer system 700 operates in the capacity of a server or as a client user computer in a server-client user network environment, or as a peer computer system in a peer-to-peer (or distributed) network environment. The computer system 700 can also be implemented as or incorporated into various devices, such as a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 700 can be incorporated as or in a device that in turn is in an integrated system that includes additional devices. In an embodiment, the computer system 700 can be implemented using electronic devices that provide voice, video or data communication. Further, while the computer system 700 is illustrated in the singular, the term “system” shall also be taken to include any collection of systems or sub-systems that individually or jointly execute a set, or multiple sets, of software instructions to perform one or more computer functions.

As illustrated in FIG. 7, the computer system 700 includes a processor 710. The processor 710 may be considered a representative example of a processor of a controller and executes instructions to implement some or all aspects of methods and processes described herein. The processor 710 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 710 is an article of manufacture and/or a machine component. The processor 710 is configured to execute software instructions to perform functions as described in the various embodiments herein. The processor 710 may be a general-purpose processor or may be part of an application specific integrated circuit (ASIC). The processor 710 may also be a microprocessor, a microcomputer, a processor chip, a controller, a microcontroller, a digital signal processor (DSP), a state machine, or a programmable logic device. The processor 710 may also be a logical circuit, including a programmable gate array (PGA), such as a field programmable gate array (FPGA), or another type of circuit that includes discrete gate and/or transistor logic. The processor 710 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 700 further includes a main memory 720 and a static memory 730, where memories in the computer system 700 communicate with each other and the processor 710 via a bus 708. Either or both of the main memory 720 and the static memory 730 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 720 and the static memory 730 are articles of manufacture and/or machine components. The main memory 720 and the static memory 730 are computer-readable mediums from which data and executable software instructions can be read by a computer (e.g., the processor 710). Each of the main memory 720 and the static memory 730 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 700 further includes a video display unit 750, such as a liquid crystal display (LCD), an organic light emitting diode (OLED), a flat panel display, a solid-state display, or a cathode ray tube (CRT), for example. Additionally, the computer system 700 includes an input device 760, such as a keyboard/virtual keyboard or touch-sensitive input screen or speech input with speech recognition, and a cursor control device 770, such as a mouse or touch-sensitive input screen or pad. The computer system 700 also optionally includes a disk drive unit 780, a signal generation device 790, such as a speaker or remote control, and/or a network interface device 740.

In an embodiment, as depicted in FIG. 7, the disk drive unit 780 includes a computer-readable medium 782 in which one or more sets of software instructions 784 (software) are embedded. The sets of software instructions 784 are read from the computer-readable medium 782 to be executed by the processor 710. Further, the software instructions 784, when executed by the processor 710, perform one or more steps of the methods and processes as described herein. In an embodiment, the software instructions 784 reside all or in part within the main memory 720, the static memory 730 and/or the processor 710 during execution by the computer system 700. Further, the computer-readable medium 782 may include software instructions 784 or receive and execute software instructions 784 responsive to a propagated signal, so that a device connected to a network 701 communicates voice, video or data over the network 701. The software instructions 784 may be transmitted or received over the network 701 via the network interface device 740.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

In some embodiments based on the teachings herein, after several progressions of the workflow in FIG. 4, if the clinician ends up repeatedly selecting the same ultrasound settings for the scan, the ultrasound system 100A or the ultrasound system 100B may automatically capture the repeated ultrasound settings as a feature-specific default combination of settings. The user may then not require progressing through the entire workflow for every exam and the clinician may instead simply select that TSP at the start of an examination. If at any point during the exam the clinician wants to evaluate/compare with the results in other settings, the progression process may be easily initiated via a button on the user interface.

Accordingly, settings progression for ultrasound provides an automated approach to highlight images based on optimal settings to the user, and this may enhance standardization and consistency in interpretation of ultrasound features such as lung ultrasound features.

Although settings progression for ultrasound has been described with reference to several exemplary embodiments, it is understood that the words that have been used are words of description and illustration, rather than words of limitation. Changes may be made within the purview of the appended claims, as presently stated and as amended, without departing from the scope and spirit of settings progression for ultrasound in its aspects. Although settings progression for ultrasound has been described with reference to particular means, materials and embodiments, settings progression for ultrasound is not intended to be limited to the particulars disclosed; rather settings progression for ultrasound extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

We claim:

1. An ultrasound system, comprising:
an ultrasound probe that transmits ultrasound imaging beams in accordance with combinations of settings;
a display that displays images based on feedback generated from the ultrasound imaging beams; and
an ultrasound base interfaced with the ultrasound probe and the display, wherein the ultrasound system is configured to automatically progress through a plurality of combinations of settings for transmitting the ultrasound imaging beams.

2. The ultrasound system of claim 1, further comprising:
a controller comprising a memory that stores instructions and a processor that executes the instructions, wherein the controller is configured to execute the instructions to control the ultrasound probe to transmit ultrasound imaging beams by automatically progressing through the plurality of combinations of settings.

3. The ultrasound system of claim 2,
wherein the controller is implemented in the ultrasound probe.

4. The ultrasound system of claim 1,
wherein the combinations of settings include a first setting for image depth, a second setting for harmonics, a third setting for a gain as a function of image depth, and a fourth setting for focal depth.

5. The ultrasound system of claim 1, wherein the display is configured to simultaneously display a first image based on feedback from a first combination of settings and a second image based on feedback from a second combination of settings.

6. The ultrasound system of claim 1, wherein the ultrasound system is configured to adaptively update the plurality of combinations of settings during a single progression.

7. The ultrasound system of claim 1, wherein the ultrasound system is configured to adaptively update a range of parameter values for each parameter in at least one setting.

8. A method of operation for an ultrasound system, the method comprising:
transmitting, by an ultrasound probe, a first ultrasound imaging beam in accordance with a first combination of settings;
displaying, by a display, a first image based on feedback generated from the first ultrasound imaging beam;
transmitting, by the ultrasound probe, a second ultrasound imaging beam in accordance with a second combination of settings;
displaying, by the display, a second image based on feedback generated from the second ultrasound imaging beam; and
automatically progressing through a plurality of combinations of settings including the first combination of settings and the second combination of settings.

9. The method of claim 8, further comprising:
executing, by a controller comprising a memory that stores instructions and a processor that executes the instructions, the instructions to control the ultrasound probe to transmit ultrasound imaging beams by automatically progressing through a plurality of combinations of settings including the first combination of settings and the second combination of settings.

10. The method of claim 9, wherein the controller is implemented in the ultrasound probe.

11. The method of claim 8, wherein the combinations of settings include a first setting for image depth, a second setting for harmonics, a third setting for a gain as a function of image depth, and a fourth setting for focal depth.

12. The method of claim 8, wherein the display is configured to simultaneously display the first image and the second image.

13. The method of claim 8, further comprising:
adaptively updating the plurality of combinations of settings during a single progression.

14. The method of claim 8, further comprising:
adaptively updating a range of parameter values for each parameter in at least one setting.

15. The method of claim 8, further comprising:
setting a default combination of settings based on selections of images generated from automatically progressing through the plurality of combinations of settings.

16. A controller, comprising:
a memory that stores instructions; and
a processor that executes the instructions;
wherein, when executed by the processor, the instructions cause the controller to:
control transmission, by an ultrasound probe, of a first ultrasound imaging beam in accordance with a first combination of settings in an automated progression;
control display, by a display, of a first image based on feedback generated from the first ultrasound imaging beam;
control transmission, by the ultrasound probe, of a second ultrasound imaging beam in accordance with a second combination of settings in the automated progression;
control display, by the display, of a second image based on feedback generated from the second ultrasound imaging beam; and
automatically progress through a plurality of combinations of settings for transmitting a plurality of ultrasound imaging beams including the first ultrasound imaging beam and the second ultrasound imaging beam.

17. The controller of claim 16,
wherein the controller is implemented in the ultrasound probe.

18. The controller of claim 16, wherein the combinations of settings include a first setting for image depth, a second setting for harmonics, a third setting for a gain as a function of image depth, and a fourth setting for focal depth.

19. The controller of claim 16, wherein, when executed by the processor, the instructions cause the controller further to:
adaptively update the plurality of combinations of settings during a single progression through the plurality of combinations of settings.

20. The controller of claim 19, wherein the single progression through the plurality of combinations of settings is performed at each of a plurality of positions of the ultrasound probe.
